(19) United States
(12) Patent Application Publication
(10) Pub. No.: US 2003/0008923 A1
(43) Pub. Date: Jan. 9, 2003

(54) Antineoplastic combinations

(75) Inventors: Gary Dukart, Ambler, PA (US); James Joseph Gibbons JR., Westwood, NJ (US)

Correspondence Address: Arnold S. Milowsky, 5 Giralda Farms, Madison, NJ, 07940, US

(73) Assignee: Wyeth, Madison, NJ (US)

(21) Appl. No.: 10/157,656

(22) Filed: May 29, 2002

Related U.S. Application Data
(60) Provisional application No. 60/295,190, filed on Jun. 1, 2001.

(51) Int. Cl. A61K 31/44; A61K 31/13

(52) U.S. Cl. 514/672

(57) ABSTRACT

This invention provides the use of a combination of an mTOR inhibitor and an antineoplastic alkylating agent in the treatment of neoplasms.

### BACKGROUND OF THE INVENTION

This application claims priority from copending provisional application Serial No. 60/295,190, filed Jun. 1, 2001, the entire disclosure of which is hereby incorporated by reference.

This invention relates to the use of combinations of an mTOR inhibitor and an alkylating agent in the treatment of neoplasms.

Rapamycin is a macrocyclic triene antibiotic produced by Streptomyces hygroscopicus, which was found to have antifungal activity, particularly against Candida albicans, both in vitro and in vivo [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. Nos. 3,929,992; and 3,993,749]. Additionally, rapamycin alone (U.S. Pat. No. 4,885,171) or in combination with picibanil (U.S. Pat. No. 4,401,653) has been shown to have antitumor activity.

The immunosuppressive effects of rapamycin have been disclosed in FASEB a* 3, 3411 (1989). Cyclosporin A and FK-506, other macrocyclic molecules, also have been shown to be effective as immunosuppressive agents, therefore useful in preventing transplant rejection [FASEB 3, 3411 (1989); FASEB 3, 5256 (1989); R. Y. Calne et al., Lancet 1183 (1978); and U.S. Pat. No. 5,100,899]. R. Martel et al. [Can. J. Physiol. Pharmacol. 55, 48 (1977)] disclosed that rapamycin is effective in the experimental allergic encephalomyelitis model, a model for multiple sclerosis; in the adjuvant arthritis model, a model for rheumatoid arthritis; and effectively inhibited the formation of IgE-like antibodies.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,009], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 A1], ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

Rapamycin 42-ester with 3-hydroxy-2-(hydroxymethyl)-2-methylpropionic acid (CCI-779) is ester of rapamycin which has demonstrated significant inhibitory effects on tumor growth in both in vitro and in vivo models. The preparation and use of hydroxyesters of rapamycin, including CCI-779, are disclosed in U.S. Pat. No. 5,362,718.

CCI-779 exhibits cytostatic, as opposed to cytotoxic properties, and may delay the time to progression of tumors or time to tumor recurrence. CCI-779 is considered to have a mechanism of action that is similar to that of sirolimus. CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S. The mechanism of action of CCI-779 that results in the G1 S phase block is novel for an anticancer drug.

In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

In vivo studies in nude mice have demonstrated that CCI-779 has activity against human tumor xenografts of diverse histological types. Gliomas were particularly sensitive to CCI-779 and the compound was active in an orthotopic glioma model in nude mice. Growth factor (platelet-derived)-induced stimulation of a human glioblastoma cell line in vitro was markedly suppressed by CCI-779. The growth of several human pancreatic tumors in nude mice as well as one of two breast cancer lines studied in vivo also was inhibited by CCI-779.

### DESCRIPTION OF THE INVENTION

This invention provides the use of combinations of an mTOR inhibitor and an antineoplastic alkylating agent as antineoplastic combination chemotherapy. In particular, these combinations are useful in the treatment of renal cancer, soft tissue cancer, breast cancer, neuroendocrine tumor of the lung, cervical cancer, uterine cancer, head and neck cancer, glioma, non-small lung cell cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. This invention also provides combinations of an mTOR inhibitor and an antineoplastic alkylating agent for use as antineoplastic combination chemotherapy, in which the dosage of either the mTOR inhibitor or the antineoplastic alkylating agent or both are used in subtherapeutically effective dosages.

As used in accordance with this invention, the term “treatment” means treating a mammal having a neoplastic disease by providing said mammal an effective amount of a combination of an mTOR inhibitor and an antineoplastic alkylating agent with the purpose of inhibiting growth of the neoplasm in such mammal, eradication of the neoplasm, or palliation of the mammal.

As used in accordance with this invention, the term “providing,” with respect to providing the combination, means either directly administering the combination, or administering a prodrug, derivative, or analog of one or both of the components of the combination which will form an effective amount of the combination within the body.

mTOR is the mammalian target of rapamycin, also known as FKBP12-rapamycin associated protein [FRAP]. Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S.

mTOR regulates the activity of at least two proteins involved in the translation of specific cell cycle regulatory proteins (Burnett, P. E., PNAS 95: 1432 (1998) and Isotani, S., J. Biol. Chem. 274: 33493 (1999)). One of these proteins p70s6 kinase is phosphorylated by mTOR on serine 389 as well as threonine 412. This phosphorylation can be observed in growth factor treated cells by Western blotting of whole cell extracts of these cells with antibody specific for the phosphoserine 389 residue.

As used in accordance with this invention, an “mTOR inhibitor” means a compound or ligand which inhibits cell replication by blocking progression of the cell cycle from G1 to S by inhibiting the phosphorylation of serine 389 of p70s6 kinase by mTOR.

The following standard pharmacological test procedure can be used to determine whether a compound is an mTOR inhibitor, as defined herein. Treatment of growth factor stimulated cells with an mTOR inhibitor like rapamycin completely blocks phosphorylation of serine 389 as evidenced by Western blot and as such constitutes a good assay for mTOR inhibition. Thus whole cell lysates from cells stimulated by a growth factor (eg. IGF1) in culture in the presence of an mTOR inhibitor should fail to show a band on an acrylamide gel capable of being labeled with an antibody specific for serine 389 of p70s6K.

Materials:

| | |
|---|---|
| NuPAGE LDS Sample Buffer | (Novex Cat # NP0007) |
| NuPAGE Sample Reducing Agent | (Novex Cat # NP0004) |
| NuPAGE 4-12% Bis-Tris Gel | (Novex Cat # NP0321) |
| NuPAGE MOPS SDS Running Buffer | (Novex Cat # NP0001) |
| Nitrocellulose | (Novex Cat # LC2001) |
| NuPAGE Transfer Buffer | (Novex Cat # NP0006) |
| Hyperfilm ECL | (Amersham Cat # RPN3114H) |
| ECL Western Blotting Detection Reagent | (Amersham Cat # RPN2134) |
| Primary antibody: Phospho-p70 S6 Kinase (Thr389) | (Cell Signaling Cat # 9205) |
| Secondary antibody: Goat anti-rabbit IgG-HRP conjugate | (Santa Cruz Cat # sc-2004) |

Methods

A. Preparation of Cell Lysates

Cell lines were grown in optimal basal medium supplemented with 10% fetal bovine serum and penicillin/treptomycin. For phosphorylation studies, cells were subcultured in 6-well plates. After the cells have completely attached, they were either serum-starved. Treatment with mTOR inhibitors ranged from 2 to 16 hours. After drug treatment, the cells were rinsed once with PBS (phosphate buffered saline without Mg++ and Ca++) and then lysed in 150-200 μl NuPAGE LDS sample buffer per well. The lysates were briefly sonicated and then centrifuged for 15 minutes at 14000 rpm. Lysates were stored at minus -80° C. until use.

The test procedure can also be run by incubating the cells in growth medium overnigh, after they have completely attached. The results under both sets of conditions should be the same for an mTOR inhibitor.

B. Western Blot Analysis

1) Prepare total protein samples by placing 22.5 μl of lysate per tube and then add 2.5 μl NuPAGE sample reducing agent. Heat samples at 70° C. for 10 minutes. Electrophoresed using NuPAGE gels and NuPAGE SDS buffers.

2) Transfer the gel to a nitrocellulose membrane with NuPAGE transfer buffer. The membrane are blocked for 1 hour with blocking buffer (Tris buffered saline with 0.1%-Tween and 5% nonfat-milk). Rinse membranes 2× with washing buffer (Tris buffered saline with 0.1%-Tween).

3) Blots/membrane are incubated with the P-p70 S6K (T389) primary antibody (1:1000) in blocking buffer overnight at 4° C. in a rotating platform.

4) Blots are rinsed 3× for 10 minutes each with washing buffer, and incubated with secondary antibody (1:2000) in blocking buffer for 1 hour at room temperature.

5) After the secondary antibody binding, blots are washed 3× for 10 minutes each with washing buffer, and 2× for 1 minute each with Tris-buffered saline, followed by chemiluminescent (ECL) detection and then exposed to chemiluminescence films.

As used in accordance with this invention, the term “a rapamycin” defines a class of immunosuppressive compounds which contain the basic rapamycin nucleus (shown below). The rapamycins of this invention include compounds which may be chemically or biologically modified as derivatives of the rapamycin nucleus, while still retaining immunosuppressive properties. Accordingly, the term “a rapamycin” includes esters, ethers, oximes, hydrazones, and hydroxylamines of rapamycin, as well as rapamycins in which functional groups on the rapamycin nucleus have been modified, for example through reduction or oxidation. The term “a rapamycin” also includes pharmaceutically acceptable salts of rapamycins, which are capable of forming such salts, either by virtue of containing an acidic or basic moiety.

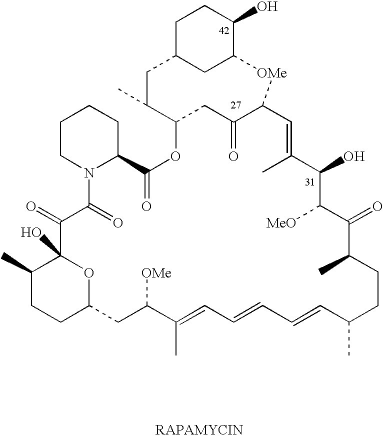

It is preferred that the esters and ethers of rapamycin are of the hydroxyl groups at the 42- and/or 31-positions of the rapamycin nucleus, esters and ethers of a hydroxyl group at the 27-position (following chemical reduction of the 27-ketone), and that the oximes, hydrazones, and hydroxylamines are of a ketone at the 42-position (following oxidation of the 42-hydroxyl group) and of 27-ketone of the rapamycin nucleus.

Preferred 42- and/or 31-esters and ethers of rapamycin are disclosed in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above.

Preferred 27-esters and ethers of rapamycin are disclosed in U.S. Pat. No. 5,256,790, which is hereby incorporated by reference. The preparation of these esters and ethers are disclosed in the patents listed above.

Preferred oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines are disclosed in the above listed patents. The preparation of 42-oxorapamycin is disclosed in U.S. Pat. No. 5,023,263, which is hereby incorporated by reference.

Particularly preferred rapamycins include rapamycin [U.S. Pat. No. 3,929,992], CCI-779 [rapamycin 42-ester with 3-hydroxy-2-(hydroxymethyl)-2-methylpropionic acid; U.S. Pat. No. 5,362,718], and 42-O-(2-hydroxy)ethyl rapamycin [U.S. Pat. No. 5,665,772].

When applicable, pharmaceutically acceptable salts of the rapamycin can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable aids when the rapamycin contains a suitable basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when the rapamycin contains a suitable acidic moiety.

It is preferred that the mTOR inhibitor used in the antineoplastic combinations of this invention is a rapamycin, and more preferred that the mTOR inhibitor is rapamycin, CCI-779, or 42-O-(2-hydroxy)ethyl rapamycin.

As described herein, CCI-779 was evaluated as a representative mTOR inhibitor in the mTOR inhibitor plus antimetabolite combinations of this invention.

The preparation of CCI-779 is described in U.S. Pat. No. 5,362,718, which is hereby incorporated by reference. When CCI-779 is used as an antineoplastic agent, it is projected that initial i.v. infusion dosages will be between about 0.1 and 100 mg/m² when administered on a daily dosage regimen (daily for 5 days, every 2-3 weeks), and between about 0.1 and 1000 mg/m² when administered on a once weekly dosage regimen. Oral or intravenous infusion are the preferred routes of administration, with intravenous being more preferred.

As used in accordance with this invention, the term “antineoplastic alkylating agent” means a substance which reacts with (or “alkylates”) many electron-rich atoms in cells to form covalent bonds. The most important reactions with regard to their antitumor activities are reactions with DNA bases. Some alkylating agents are monofunctional and react with only one strand of DNA. Others are bifunctional and react with an atom on each of the two strands of DNA to produce a “cross-link” that covalently links the two strands of the DNA double helix. Unless repaired, this lesion will prevent the cell from replicating effectively. The lethality of the monofunctional alkylating agents results from the recognition of the DNA lesion by the cell and the response of the cell to that lesion. (Colvin O M. Antitumor Alkylating Agents. In Cancer Principles & Practice of Oncology 6^{th} Edition. ed. DeVita V T, Hellman S, Rosenberg S A. Lippincoft Williams & Wilkins. Philadelphia 2001. p. 363.)

Antineoplastic alkylating agents are roughly classified, according to their structure or reactive moiety, into several categories which include nitrogen mustards, such as mustargen, cyclophosphamide, ifosfamide, melphalan, and chlorambucil; azidines and epoxides, such as thiotepa, mitomycin C, dianhydrogalactitol, and dibromodulcitol; alkyl sulfonates, such as busulfan; nitrosoureas, such as bischloroethylnitrosourea (BCNU), cyclohexyl-chloroethylnitrosourea (CCNU), and methylcyclohexylchloroethylnitrosourea (MeCCNU); hydrazine and triazine derivatives, such as procarbazine, dacarbazine, and temozolomide; and platinum compounds. Platinum compounds are platinum containing agents that react preferentially at the N7 position of guanine and adenine residues to form a variety of monofunctional and bifunctional adducts. (Johnson S W, Stevenson J P, O'Dwyer P J. Cisplatin and Its Analogues. In Cancer Principles & Practice of Oncology 6^{th} Edition. ed. DeVita V T, Hellman S, Rosenberg S A. Lippincott Williams & Wilkins. Philadelphia 2001. p. 378.) These compounds include cisplatin, carboplatin, platinum IV compounds, and multinuclear platinum complexes.

The following are representative examples of alkylating agents of this invention.

Meclorethamine is commercially available as an injectable (MUSTARGEN).

Cyclophosphamide is commercially available as an injectable (cyclophosphamide, lyophilized CYTOXAN, or NEOSAR) and in oral tablets (cyclophosphamide or CYTOXAN).

Ifosfamide is commercially available as an injectable (IFEX).

Melphalan is commercially available as an injectable (ALKERAN) and in oral tablets (ALKERAN).

Chlorambucil is commercially available in oral tablets (LEUKERAN).

Thiotepa is commercially available as an injectable (thiotepa or THIOPLEX).

Mitomycin is commercially available as an injectable (mitomycin or MUTAMYCIN).

Busulfan is commercially available as an injectable (BUSULFEX) and in oral tablets (MYLERAN).

Lomustine (CCNU) is commercially available in oral capsules (CEENU).

Carmustine (BCNU) is commercially available as an intracranial implant (GLIADEL) and as an injectable (BICNU).

Procarbazine is commercially available in oral capsules (MATULANE).

Temozolomide is commercially available in oral capsules (TEMODAR).

Cisplatin is commercially available as an injectable (cisplatin, PLATINOL, or PLATINOL-AQ).

Carboplatin is commercially available as an injectable (PARAPLATIN).

The following table briefly summarizes some of the recommended dosages for the antineoplastic alkylating agents listed above.

TABLE 1
Recommended Dosages of Antineoplastic Alkylating Agents

| Drug | Dosage | | Regimen |
|---|---|---|---|
| Mustargen | 0.4 | mg/kg | each course given as a singe dose or in |
| | | | divided doses of 0.1 to 0.2 mg/kg/day. |
| Cyclophosphamide | 40-50 | mg/kg i.v. | in divided doses over a period of 2-5 days |
| | 10-15 | mg/kg i.v. | every 7-10 days |
| | 3-5 | mg/kg i.v. | twice weekly |
| | 1-5 | mg/kg oral | daily |
| Ifosfamide | 1.2 | g/m² i.v. | daily for 5 consecutive days; repeated |
| | | | every 3 weeks or after recovery from |
| | | | hematologic toxicity. |
| Melphalan | 6 | mg orally | daily for 2-3 weeks followed by 4 weeks |
| | | | rest, then 2 mg daily maintenance dosage |
| | 10 | mg orally | daily for 7-10 days followed by 2 mg daily |
| | | | maintenance after white blood cell count |
| | | | has recovered. |
| | 0.15 | mg/kg orally | daily for 7 days, followed by a rest period |
| | | | of at least 14 days, then 0.005 mg/kg |
| | | | daily maintenance. |
| | 16 | mg/m² i.v. | single infusion over 15-20 minutes every |
| | | | 2 weeks for 4 doses, followed by a rest |
| | | | period, then administered at 4 week |
| | | | intervals for maintenance. |
| Chlorambucil | 0.1-0.2 | mg/kg orally | daily for 3-6 weeks |
| Thiotepa | 0.3-0.4 | mg/kg i.v. | every 1-4 weeks |
| Mitomycin | 20 | mg/m² i.v. | every 6-8 weeks |
| Busulfan | 1.8 | mg/m² orally | daily |
| Lomustine | 130 | mg/m² orally | every 6 weeks |
| Carmustine | 150-200 | mg/m² i.v. | every 6 weeks |
| Procarbazine | 2-4 | mg/kg orally | daily for first week, then 4-6 mg/kg until |
| | | | maximum response is achieved |
| | 1-2 | mg/kg orally | mainentance |
| Temozolomide | 150 | mg/m² orally | once daily for 5 days per 28-day |
| | | | treatment cycle |
| Cisplatin | 20 | mg/m² i.v. | daily for 5 days per cycle |
| | 75-100 | mg/m² i.v. | once every 4 week cycle |
| Carboplatin | 360 | mg/m² i.v. | once every 4 week cycle |

Preferred mTOR inhibitor plus antineoplastic alkylating agent combinations of this invention include CCI-779 plus cisplatin; CCI-779 plus cyclophosphamide; CCI-779 plus carboplatin; and CCI-779 plus BCNU.

The antineoplastic activity of the mTOR inhibitor plus antineoplastic alkylating agent combinations were confirmed using CCI-779 as a representative mTOR inhibitor in in vitro and in vivo standard pharmacological test procedures using combinations of CCI-779 plus cisplatin; CCI-779 plus cyclophosphamide; and CCI-779 plus BCNU as representative combinations of this invention. The following briefly describes the procedures used and the results obtained.

Human rhabdomyosarcoma lines Rh30 and Rh1 and the human glioblastoma line SJ-GBM2 were used for in vitro combination studies with CCI-779 and alkylating agents. In vivo studies used a human neuroblastoma (NB1643) and human colon line GC3.

Dose response curves were determined for each of the drugs of interest. The cell lines Rh30, Rh1 and SJ-G2 were plated in six-well cluster plates at 6×10³, 5×10³ and 2.5×10⁴ cells/well respectively. After a 24 hour incubation period, drugs were added in either 10%FBS+RPMI 1640 for Rh30 and Rh1 or 15% FBS+DME for SJ-G2. After seven days exposure to drug containing media, the nuclei were released by treating the cells with a hypotonic solution followed by a detergent. The nuclei were then counted with a Coulter Counter. The results of the experiments were graphed and the IC₅₀ (drug concentration producing 50% inhibition of growth) for each drug was determined by extrapolation. Because the IC50s varied slightly from experiment to experiment, two values that bracketed the IC50 of each drug were used in the interaction studies. The point of maximum interaction between two drugs occurs when they are present in a 1:1 ratio if the isobole is of standard shape. Therefore, each of the three approximate IC₅₀ concentrations of CCI-779 was mixed in a 1:1 ratio with each of three approximated IC₅₀s of cisplatin, BCNU, and melphanan. This resulted in nine 1:1 combinations of drugs in each experiment plus three IC₅₀ concentrations for CCI-779 and the other drug. This protocol usually resulted in at least one combination for each drug containing an IC₅₀ value. The 1:1 combination of IC₅₀ concentrations for CCI-779 and each chemotherapy drug was then used to calculate additivity, synergism, or antagonism using Berenbaum's formula: x/X₅₀+y/Y₅₀,=1,<1,>1. If the three concentrations of CCI-779 tested alone didn't produce an IC that matched any of the three ICs of the other compound tested alone, all the 1:1 combinations were checked to see if their ICs fell between the appropriate ICs of drugs tested singly. If they did, the effect was considered additive.

The results obtained in the in vitro standard pharmacological test procedure showed when tested against Rh30 tumor line, the combination of CCI-779 plus cisplatin was synergistic; the combination was greater than additive but did not reach levels of being mathematically synergystic against the Rh1 tumor cell line, and was additive against the SJ-G2 tumor cell line. A combination of CCI-779 plus BCNU was synergistic against the SJ-G2 tumor cell line and greater than additive but did not reach levels of being mathematically synergystic against the Rh30 cell line, and additive against the Rh1 cell line. The combination of CCI-779 plus melphanan was additive against each of the cell lines.

Female CBA/CaJ mice (Jackson Laboratories, Bar Harbor, Me.), 4 weeks of age, were immune-deprived by thymectomy, followed 3 weeks later by whole-body irradiation (1200 cGy) using a ¹³⁷Cs source. Mice received 3×10⁶ nucleated bone marrow cells within 6-8 h of irradiation. Tumor pieces of approximately 3 mm³ were implanted in the space of the dorsal lateral flanks of the mice to initiate tumor growth. Tumor-bearing mice were randomized into groups of seven prior to initiating therapy. Mice bearing tumors each received drug when tumors were approximately 0.20-1 cm in diameter. Tumor size was determined at 7-day intervals using digital Vernier calipers interfaced with a computer. Tumor volumes were calculated assuming tumors to be spherical using the formula [(π/6)×d³], where d is the mean diameter. CCI-779 was given on a schedule of 5 consecutive days for 2 weeks with this cycle repeated every 21 days for 3 cycles. This resulted in CCI-779 being given on days 1-5, 8-12 (cycle 1); 21-25, 28-32 (cycle 2); and 42-46, 49-53 (cycle 3). The schedule of the other chemotherapy drug for each study was as follows:

Cyclophosphamide on days 1 and 8 every 21 days for 3 cycles

The combination of CCI-779 and cyclophosphamide was evaluated using a human rhabdosarcoma (Rh18) using the mouse xenograft test procedure described above. In this test procedure, the effect of CCI-779 with cyclophosphamide (44 mg/kg) was additive. When combined as suboptimum dosages, CCI-779 plus cyclophosphamide was equivalent to cyclophosphamide given at an optimum dosage.

Based on the results of these standard pharmacological test procedures, combinations of an mTOR inhibitor plus an antineoplastic alkylating agent are useful as antineoplastic therapy. More particularly, these combinations are useful in the treatment of renal carcinoma, soft tissue sarcoma, breast cancer, neuroendocrine tumor of the lung, cervical cancer, uterine cancer, head and neck cancer, glioma, non-small cell lung cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. As these combinations contain at least two active antineoplastic agents, the use of such combinations also provides for the use of combinations of each of the agents in which one or both of the agents is used at subtherapeutically effective dosages, thereby lessening toxicity associated with the individual chemotherapeutic agent.

In providing chemotherapy, multiple agents having different modalities of action are typically used as part of a chemotherapy “cocktail.” It is anticipated that the combinations of this invention will be used as part of a chemotherapy cocktail that may contain one or more additional antineoplastic agents depending on the nature of the neoplasia to be treated. For example, this invention also covers the use of the mTOR inhibitor/alkylating agent combination used in conjunction with other chemotherapeutic agents, such as antimetabolites (i.e., 5-fluorouracil, floxuradine, thioguanine, cytarabine, fludarabine, 6-mercaptopurine, methotrexate, gemcitabine, capecitabine, pentostatin, trimetrexate, or cladribine); hormonal agents (i.e., estramustine, tamoxifen, toremifene, anastrozole, or letrozole); antibiotics (i.e., plicamycin, bleomycin, mitoxantrone, idarubicin, dactinomycin, mitomycin, or daunorubicin); immunomodulators (i.e., interferons, IL-2, or BCG); antimitotic agents (i.e., vinblastine, vincristine, teniposide, or vinorelbine); topoisomerase inhibitors (i.e., topotecan, irinotecan, etoposide, or doxorubicin); and other agents (i.e., hydroxyurea, trastuzumab, altretamine, retuximab, paclitaxel, docetaxel, L-asparaginase, or gemtuzumab ozogamicin).

As used in this invention, the combination regimen can be given simultaneously or can be given in a staggered regimen, with the mTOR inhibitor being given at a different time during the course of chemotherapy than the alkylating agent. This time differential may range from several minutes, hours, days, weeks, or longer between administration of the two agents. Therefore, the term combination does not necessarily mean administered at the same time or as a unitary dose, but that each of the components are administered during a desired treatment period. The agents may also be administered by different routes. For example, in the combination of an mTOR inhibitor plus an alkylating agent, it is anticipated that the mTOR inhibitor will be administered orally or parenterally, with parenterally being preferred, while the alkylating agent may be administered parenterally, orally, or by other acceptable means. These combination can be administered daily, weekly, or even once monthly. As typical for chemotherapeutic regimens, a course of chemotherapy may be repeated several weeks later, and may follow the same timeframe for administration of the two agents, or may be modified based on patient response.

As typical with chemotherapy, dosage regimens are closely monitored by the treating physician, based on numerous factors including the severity of the disease, response to the disease, any treatment related toxicities, age, health of the patient, and other concomitant disorders or treatments.

Based on the results obtained with the CCI-779 plus alkylating agent combinations, it is projected that the initial i.v. infusion dosage of the mTOR inhibitor will be between about 0.1 and 100 mg/m², with between about 2.5 and 70 mg/m² being preferred. It is also preferred that the mTOR inhibitor be administered by i.v., typically over a 30 minute period, and administered about once per week. The initial dosages of the alkylating agent component will depend on the component used, and will be based initially on physician experience with the agents chosen. After one or more treatment cycles, the dosages can be adjusted upwards or downwards depending on the results obtained and the side effects observed.

For commercially available alkylating agents, the existing dosage form can be used, with the dosages divided as need be. Alternatively, such agents or alkylating agents that are not commercially available can be formulated according to standard pharmaceutical practice. Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

What is claimed is:

1. A method of treating a neoplasm in a mammal in need thereof, which comprises providing to said mammal an effective amount of a combination comprising an mTOR inhibitor and an antineoplastic alkylating agent.

2. The method according to claim 1, wherein the neoplasm is renal cancer.

3. The method according to claim 1, wherein the neoplasm is soft tissue sarcoma.

4. The method according to claim 1, wherein the neoplasm is breast cancer.

5. The method according to claim 1, wherein the neoplasm is a neuroendocrine tumor of the lung.

6. The method according to claim 1, wherein the neoplasm is cervical cancer.

7. The method according to claim 1, wherein the neoplasm is uterine cancer.

8. The method according to claim 1, wherein the neoplasm is a head and neck cancer.

9. The method according to claim 1, wherein the neoplasm is glioma.

10. The method according to claim 1, wherein the neoplasm is non-small cell lung cancer.

11. The method according to claim 1, wherein the neoplasm is prostate cancer.

12. The method according to claim 1, wherein the neoplasm is pancreatic cancer.

13. The method according to claim 1, wherein the neoplasm is lymphoma.

14. The method according to claim 1, wherein the neoplasm is melanoma.

15. The method according to claim 1, wherein the neoplasm is small cell lung cancer.

16. The method according to claim 1, wherein the neoplasm is ovarian cancer.

17. The method according to claim 1, wherein the neoplasm is colon cancer.

18. The method according to claim 1, wherein the neoplasm is esophageal cancer.

19. The method according to claim 1, wherein the neoplasm is gastric cancer.

20. The method according to claim 1, wherein the neoplasm is leukemia.

21. The method according to claim 1, wherein the neoplasm is colorectal cancer.

22. The method according to claim 1, wherein the neoplasm is unknown primary cancer.

23. The method according to claim 1, wherein the antineoplastic alkylating agent is selected from the group consisting of meclorethamine, cyclophosphamide, ifosfamide, melphalan, chlorambucil, thiotepa, mitomycin, busulfan, lomustine, carmustine, procarbazine, temozolomide, cisplatin, and carboplatin.

24. A method of treating a neoplasm in a mammal in need thereof, which comprises providing to said mammal an effective amount of a combination comprising an mTOR inhibitor and an antineoplastic alkylating agent, wherein either the mTOR inhibitor, the alkylating agent, or both are provided in subtherapeutically effective amounts.

25. The method according to claim 24 in which the mTOR inhibitor is provided in a subtherapeutically effective amount.

26. The method according to claim 24 in which the alkylating agent is provided in a subtherapeutically effective amount.

27. The method according to claim 24 in which both the mTOR inhibitor and the alkylating agent are provided in subtherapeutically effective amounts.

28. The method according to claim 1, wherein the mTOR inhibitor is a rapamycin.

29. The method according to claim 28, wherein the rapamycin is rapamycin.

30. The method according to claim 28, wherein the rapamycin is 42-O-(2-hydroxy)ethyl rapamycin.

31. An antineoplastic combination which comprises an effective amount of an mTOR inhibitor and an antineoplastic alkylating agent.

32. The combination according to claim 31, wherein the mTOR inhibitor is a rapamycin.

33. The combination according to claim 31, wherein the rapamycin is rapamycin.

34. The combination according to claim 32, wherein the rapamycin is 42-O-(2-hydroxy)ethyl rapamycin.
